(19) United States
(12) Patent Application Publication
(10) Pub. No.: US 2006/0233666 A1
(43) Pub. Date: Oct. 19, 2006

(54) Visual display for meter testing bodily fluids

(75) Inventors: Sonny Vu, Cambridge, MA (US); Gary Tsai, Cambridge, MA (US); Joseph Flaherty, Westford, MA (US); Stuart Blitz, Cambridge, MA (US); Eileen Huang, Hillsborough, NJ (US)

Correspondence Address: OPPEDAHL AND LARSON LLP, P O BOX 5068, DILLON, CO 80435-5068, CO, 80435-5068, US

(73) Assignee: AgaMatrix, Inc., Cambridge, MA (US)

(21) Appl. No.: 10/907,807

(22) Filed: Apr. 15, 2005

(51) Int. Cl. G01N 33/48; G01N 33/487

(52) U.S. Cl. 422/68.1; 204/403.01

(57) ABSTRACT

Abstract of the Disclosure

A test meter has a display, and has a test strip containing an electrochemical cell disposed to analyze a bodily fluid for a property of interest. While the analysis is in progress, the display is changed no less often than once per half second.

### Detailed Description of the Invention

### Background

Glucose test meters for use by lay persons are well known. Such meters are a special case of a more general category of meters testing bodily fluids for properties of interest. The users of such meters fall along a wide spectrum in terms of behavior, with some of the users being more or less diligent than others in performing tests as often as they should.

In the particular case of blood glucose meters, the meter is typically being used to attempt to control Type I diabetes. Such control depends for its chances of success on the user being diligent about performing glucose measurements at prescribed times.

Test meters are made by many different makers, and they differ in terms of how long they require to perform the desired analysis. Some of the fastest blood glucose meters are able to arrive at their conclusions in as little as a few seconds, while other blood glucose meters take much longer to do so.

Human factors such as impatience can cause problems. For one example of such a human factor, consider that if a user comes to be accustomed to the feeling that the meter does not arrive at its result promptly, there is the risk that such a user may lose interest in the testing and not perform the testing as often as directed. For a second example, a user faced with numerous makes and models of blood glucose meters may well choose unwisely among the makes and models, choosing a less accurate meter simply because it is perceived as finding its res ults quickly. There is thus a great need for blood glucose meters in particular, and for other meters generally, that would be unlikely to discourage a user with the impression that the meter is slow. There is likewise a great need for blood glucose meters in particular, and for other meters generally, that would be of high quality and that would not prompt the user to select unwisely because of perceived slowness of a particular meter.

### Summary

A test meter has a display, the test meter having a test strip containing an electrochemical cell disposed to analyze a bodily fluid for a property of interest. The test begins by commencing analysis of the sample of the bodily fluid. while the analysis is in progress, the display is changed no less often than once per half second throughout the analysis. When the analysis is almost finished, the display changes more often.

### Description of the drawing

The invention will be described with respect to a drawing in several figures, of which:
Fig. 1 shows a typical display;
Fig. 2 shows a sequence of steps.

### Detailed description

Fig. 1 shows a typical display on a test meter. It has a date field 11, a time field 12, large digits 13, 14 for providing glucose measurement results for a human user, and a progress bar area 15.

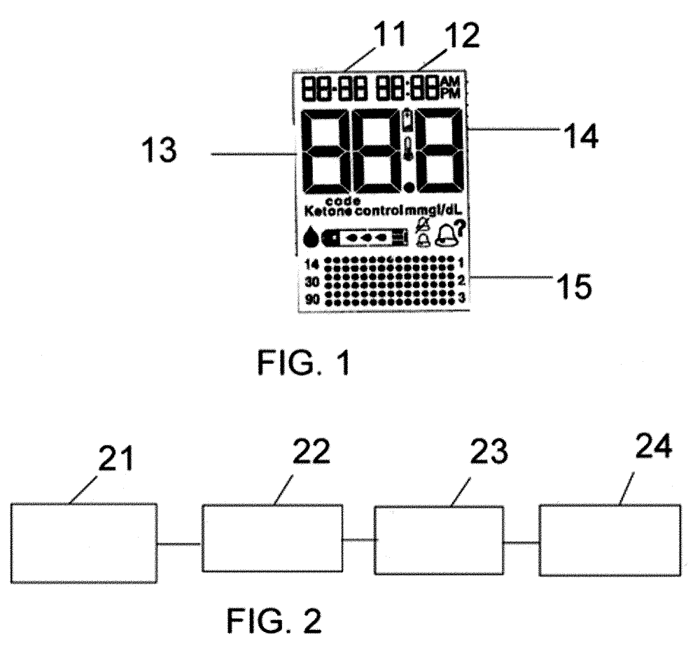

Fig. 2 shows a sequence of steps. The analysis begins at 21, for example if a user inserts a test strip into a meter and gives a drop of blood to the test strip.

During the time that follows (e.g. box 22), the display is kept nearly constantly in motion, and in any event the display is changed in some way at least once per second. This may be done by cycling through a pattern of displaying and withholding date 11 and time 12 information. Progress bars or patterns in a region 15 may also be turned on or off or used to display patterns evocative of progress.

When the analysis has only about one second left (box 23), the pattern of change of the display increases. This may comprise applying faster-moving patterns to the region 15. The meter displays its results in box 24 (Fig. 2) using digits 13, 14 (Fig. 1).

In this way a human user is more likely to remain engaged in the process of analysis and is more likely to be diligent about performing tests as directed. In the case where other meters that are perceived as being as fast as or faster than a particular model of meter are also less accurate than that particular model of meter, the invention may help to retain users of a more accurate meter.

It should also be appreciated that the invention is not limited to the methods disclosed above, but also the apparatus required in the performance of the methods.

Those skilled in the art will have no difficulty devising myriad obvious improvements and variations, all of which are intended to fall within the scope of the invention as defined by the claims that follow.

What is Claimed is:

1. A method for use with a test meter, the test meter having a display, the test meter having a test strip containing an electrochemical cell disposed to analyze a bodily fluid for a property of interest, the method comprising the steps of:
responding to application of the bodily fluid to the electrochemical cell by commencing an analysis of the bodily fluid for the property of interest; and
while the an alysis is in progress, changing the display no less often than once per half second throughout the analysis.

2. The method of claim 1 further comprising the step of:
accelerating the rate of change of the display during the last second of analysis.

3. The method of claim 1 wherein the display includes date and time display elements, and wherein the changing of the display includes turning the date display elements on and off, and turning the time display elements on and off.

4. The method of claim 1 wherein the display comprises progress bars, the method further characterized in that changing the display comprises dynamically varying the rate of change of the progress bars while the analysis is taking place.

5. The method of claim 4 wherein the varying of the rate of change of the progress bars comprises speeding the rate of change of the progress bars as the analysis progresses.
